(19) United States
(12) Patent Application Publication
(10) Pub. No.: US 2026/0091240 A1
(43) Pub. Date: Apr. 2, 2026

(54) Electrode Assembly for Applying Tumor Treating Fields (TTFields) to a Subject's Body, With a Pull-Cord and/or an Adhesive Disrupting Thread to Facilitate Removal of the Electrode Assembly

(71) Applicant: Novocure GmbH, Baar (CH)

(72) Inventor: Dmitry DUBITSKY, Haifa (IL)

(73) Assignee: Novocure GmbH, Baar (CH)

(21) Appl. No.: 19/341,354

(22) Filed: Sep. 26, 2025

Related U.S. Application Data
(60) Provisional application No. 63/700,188, filed on Sep. 27, 2024.
(60) Provisional application No. 63/700,299, filed on Sep. 27, 2024.

(51) Int. Cl. A61N 1/40; A61N 1/04

(52) U.S. Cl. CPC A61N 1/40; A61N 1/0496

(57) ABSTRACT

Tumor Treating Fields (TTFields) therapy relies on self-adhesive electrode assemblies positioned on opposite sides of the subject's body to induce an alternating electric field in a target region of the subject's body. But in certain situations, it can be difficult to remove the electrode assemblies from the subject's body. Removal can be facilitated by incorporating a cord or cable affixed near the periphery of the electrode assembly, e.g., at a corner of the electrode assembly. And this cord or cable is used to peel the electrode assembly away from the subject's body. Removal can also be facilitated by incorporating one or more threads configured to slide between the front face of the electrode assembly and the subject's skin to break the adhesive grip that holds a portion of the electrode assembly to the skin.

### CROSS REFERENCE TO RELATED APPLICATIONS

This Application claims the benefit of U.S. Provisional Applications 63/700,188 (filed Sep. 27, 2024) and 63/700,299 (filed Sep. 27, 2024), each of which is incorporated herein by reference in its entirety.

### BACKGROUND

Tumor Treating Fields (TTFields) therapy is a proven approach for treating tumors using alternating electric fields at frequencies e.g., between 50 kHz-5 MHz, more commonly 100-500 KHz. The alternating electric fields are induced by electrode assemblies (also called transducer arrays) placed on the subject's skin on opposite sides of the subject's body. When an AC voltage is applied between opposing electrode assemblies, it induces an alternating electric field in a target region of the subject's body, which provides a therapeutic effect.

In the prior art Optune® system, TTFields are delivered to patients via four self-adhesive electrode assemblies that are positioned on the patient's skin. The electrode assemblies are arranged in two pairs, with one pair of electrode assemblies typically positioned to the left and right of the tumor, and the other pair of electrode assemblies typically positioned anterior and posterior to the tumor.

One issue with the prior art self-adhesive electrode assemblies is that they are only used for a limited period of time (e.g., 10-14 days), after which they must be removed (e.g., by peeling them away from the subject's body). A new set of electrode assemblies can then be applied to continue the TTFields treatment. However, due to the relatively strong nature of the adhesive that holds the electrode assemblies to the subject body, it can sometimes be difficult to remove the prior art electrode assemblies.

The level of difficulty of removal also depends on other factors besides the nature of the adhesive. For example, in the context of treating brain tumors, it is usually not too difficult to remove the self-adhesive electrode assemblies after they have been used because they can be removed by peeling them away from the subject's head (akin to the way that a similarly-shaped bandage would be removed). But in the context of treating lung cancer, pancreatic cancer, or other tumors located within the subject's torso, it can be much more difficult to remove the self-adhesive electrode assemblies because some of the electrode assemblies will be located on the subject's back. Moreover, the difficulty in removing self-adhesive electrode assemblies from a subject's back can be compounded by a number of factors. First, the electrode assemblies for treating tumors located within a subject's torso are typically much larger than the electrode assemblies that are used to treat tumors located in a subject's head. And second, while it can be difficult for even able-body people to remove a large self-adhesive item from their back, the difficulty can become insurmountable for an unassisted individual with limited mobility.

### SUMMARY OF THE INVENTION

One aspect of the invention is directed to a first apparatus for applying an electrical signal to a subject's body. The first apparatus comprises a flexible substrate, at least one conductive pad, a flexible backing, a first layer of conductive adhesive, and a first cable or cord. The flexible substrate has a front face and a rear face. The at least one conductive pad is disposed on the front face of the substrate. The flexible backing is affixed to the rear face of the flexible substrate and the flexible backing has an outer perimeter. The first layer of conductive adhesive or conductive gel is disposed in front of the at least one conductive pad, and the first layer of conductive adhesive or conductive gel is biocompatible and is configured to adhere to the subject's body. The first cable or cord is affixed to at least one of the substrate and the flexible backing at a position that is less than 3 cm away from the outer perimeter of the flexible backing. The flexibility of the substrate, the flexibility of the flexible backing, the adhesive strength of the first layer of conductive adhesive or conductive gel, the affixation strength of the first cable or cord, and the pull strength of the first cable or cord are all within respective ranges such that when the first layer of conductive adhesive or conductive gel has been adhered to the subject's body, the flexible backing, the at least one conductive pad, and the flexible substrate can all be peeled away from the subject's body by pulling on the first cable or cord.

In some embodiments of the first apparatus, the first cable or cord is affixed to at least one of the substrate and the flexible backing at a position that is less than 1.5 cm away from the outer perimeter of the flexible backing. In some embodiments of the first apparatus, the flexible backing has a first corner, and the first cable or cord is affixed to at least one of the substrate and the flexible backing at a position that is less than 3 cm away from the first corner of the flexible backing.

In some embodiments of the first apparatus, the flexible backing has a first corner, and the first cable or cord is affixed to at least one of the substrate and the flexible backing at a position that is less than 1.5 cm away from the first corner of the flexible backing. Optionally, in these embodiments, the first cable or cord can be at least 10 cm long.

In some embodiments of the first apparatus, the flexible backing has a first corner and a second corner, and the first cable or cord is affixed to at least one of the substrate and the flexible backing at a position that is less than 3 cm away from the first corner of the flexible backing. These embodiments further comprise a second cord that is affixed to at least one of the substrate and the flexible backing at a position that is less than 3 cm away from the second corner of the flexible backing. The flexibility of the substrate, the flexibility of the flexible backing, the adhesive strength of the first layer of conductive adhesive or conductive gel, the affixation strength of the second cord, and the pull strength of the second cord are all within respective ranges such that when the first layer of conductive adhesive or conductive gel has been adhered to the subject's body, the flexible backing, the at least one conductive pad, and the flexible substrate can all be peeled away from the subject's body by pulling on the second cord.

In some embodiments of the first apparatus, the first cable or cord is affixed to the flexible backing. In some embodiments of the first apparatus, the first cable or cord is affixed to the substrate.

Some embodiments of the first apparatus further comprise a flexible sheet of conductive material positioned between the at least one conductive pad and the first layer of conductive adhesive or conductive gel. In these embodiments, the flexible sheet of conductive material is disposed in electrical contact with both the at least one conductive pad and the first layer of conductive adhesive or conductive gel.

Some embodiments of the first apparatus further comprise a sheet of graphite positioned between the at least one conductive pad and the first layer of conductive adhesive or conductive gel. In these embodiments, the sheet of graphite is disposed in electrical contact with both the at least one conductive pad and the first layer of conductive adhesive or conductive gel. Optionally, these embodiments can further comprise a second layer of conductive adhesive disposed between the at least one conductive pad and the rear face of the sheet of conductive material.

Some embodiments of the first apparatus further comprise a flexible sheet of polymer material with a dielectric constant of at least 10 positioned between the at least one conductive pad and the first layer of conductive adhesive or conductive gel.

In some embodiments of the first apparatus, the first cable or cord is a first cord that is not electrically connected to the at least one conductive pad, and the apparatus further comprises a second cable that is mounted to the substrate in a manner that electrically connects at least one electrical conductor of the second cable to the at least one conductive pad.

Optionally, in the embodiments described in the previous paragraph, the second cable can be attached to the first cord less than 5 cm away from a point at which the first cord is affixed to at least one of the substrate and the flexible backing. And the second cable and the first cord are configured so that pull forces applied to the second cable in a direction that is parallel to a vector that runs from (a) the point at which the first cord is affixed to at least one of the substrate and the flexible backing to (b) a centroid of the flexible backing are transmitted to the first cord via the second-cable-to-first-cord attachment.

Optionally, in the embodiments described in the previous paragraph, the second cable is mounted to the substrate at a position that is more than 3 cm away from the outer perimeter of the flexible backing.

In some embodiments of the first apparatus, the first cable or cord is a first cable that includes at least one electrical conductor that is electrically connected to the at least one conductive pad.

Another aspect of the invention is directed to a second apparatus for applying an electrical signal to a subject's body. The second apparatus comprises a flexible sheet of graphite, a first layer of conductive adhesive, and a first cord. The flexible sheet of graphite has a front face, a rear face, and an outer perimeter. The first layer of conductive adhesive is disposed on the front face of the sheet of graphite, and the first layer of conductive adhesive is biocompatible and is configured to adhere to the subject's body. The first cord is affixed to the sheet of graphite at a position that is less than 3 cm away from the outer perimeter of the sheet of graphite. The flexibility of the sheet of graphite, the adhesive strength of the first layer of conductive adhesive, the affixation strength of the first cord, and the pull strength of the first cord are all within respective ranges such that when the first layer of conductive adhesive has been adhered to the subject's body, the sheet of graphite can be peeled away from the subject's body by pulling on the first cord.

In some embodiments of the second apparatus, the first cord is affixed to the sheet of graphite at a position that is less than 1.5 cm away from the outer perimeter of the sheet of graphite. In some embodiments of the first apparatus, the sheet of graphite has a first corner, and the first cord is affixed to the sheet of graphite at a position that is less than 3 cm away from the first corner of the sheet of graphite.

In some embodiments of the second apparatus, the sheet of graphite has a first corner, and the first cord is affixed to the sheet of graphite at a position that is less than 1.5 cm away from the first corner of the sheet of graphite. Optionally, in these embodiments, the first cord is at least 10 cm long.

In some embodiments of the second apparatus, the sheet of graphite has a first corner and a second corner, and the first cord is affixed to the sheet of graphite at a position that is less than 3 cm away from the first corner of the sheet of graphite. These embodiments further comprise a second cord that is affixed to the sheet of graphite at a position that is less than 3 cm away from the second corner of the sheet of graphite. In these embodiments, the flexibility of the sheet of graphite, the adhesive strength of the first layer of conductive adhesive, the affixation strength of the second cord, and the pull strength of the second cord are all within respective ranges such that when the first layer of conductive adhesive has been adhered to the subject's body, the sheet of graphite can be peeled away from the subject's body by pulling on the second cord.

Optionally, in the embodiments described in the previous paragraph, the first cord is affixed to the sheet of graphite at a position that is less than 1.5 cm away from the first corner of the sheet of graphite, and the second cord is affixed to the sheet of graphite at a position that is less than 1.5 cm away from the second corner of the sheet of graphite.

Some embodiments of the second apparatus further comprise a grid of reinforcing threads that is bonded to the sheet of graphite, and the first cord is affixed to the grid of reinforcing threads. Thus, in these embodiments, the first cord is affixed to the sheet of graphite indirectly via the grid of reinforcing threads.

Another aspect of the invention is directed to a first method of removing an assembly that has previously been affixed to a surface of a subject's body. The assembly includes a flexible patch that has a front surface that is coated with a conductive adhesive and a first cable or cord affixed to a first corner of the flexible patch. The first method comprises pulling the first cable or cord in a direction that deviates by <30° from parallel to the surface of the subject's body so that a first portion of the flexible patch bends back on itself, beginning at the first corner and continuing until the flexible patch has peeled away from the surface of the subject's body. The first corner and a centroid of the flexible patch get closer to each other during an initial phase of motion.

In some instances of the first method, the assembly includes a second cord affixed to a second corner of the flexible patch, and the method further comprises pulling the second cord in a direction that deviates by <30° from parallel to the surface of the subject's body so that a second portion of the flexible patch bends back on itself, beginning at the second corner and continuing until the flexible patch has peeled away from the surface of the subject's body. The second corner and the centroid get closer to each other during the initial phase of motion.

In some instances of the first method, the first cable or cord is pulled in a direction that deviates by <10° from parallel to the surface of the subject's body.

Another aspect of the invention is directed to a third apparatus for applying an electrical signal to a subject's body. The third apparatus comprises a flexible substrate, at least one conductive pad, a flexible backing, a first layer of adhesive, and a first thread. The flexible substrate has a front face and a rear face. The at least one conductive pad is disposed on the front face of the substrate. The flexible backing is affixed to the rear face of the flexible substrate, and the flexible backing has a total area and an outer perimeter. The first layer of adhesive is disposed on the front face of the flexible backing, and the first layer of adhesive is configured to adhere to the subject's body. The first thread has (a) a first portion positioned in front of the flexible backing such that when the first layer of adhesive is adhered to the subject's body, the first portion of the first thread will be disposed between the flexible backing and the subject's body and (b) at least one second portion that extends beyond the perimeter of the flexible backing. The first and second portions of the first thread are positioned with respect to the flexible backing so that pulling the at least one second portion of the first thread in a direction that is parallel to the front face of the flexible backing will cause the first portion of the first thread to move in a direction that is parallel to the front face of the flexible backing, thereby detaching at least a portion of the flexible backing from the subject's body.

In some embodiments of the third apparatus, the first portion of the first thread is positioned so that an area bounded by the first portion of the first thread and the perimeter of the flexible backing is less than 10% of the total area.

In some embodiments of the third apparatus, the first portion of the first thread is positioned so that an area bounded by the first portion of the first thread and the perimeter of the flexible backing is less than 10% of the total area, the flexible backing has a first corner, and the area bounded by the first portion of the first thread and the perimeter of the flexible backing is located at the first corner.

Optionally, the embodiments described in the previous paragraph further comprise a cord that is affixed to at least one of the substrate and the flexible backing at a position that is less than 3 cm away from the first corner. In these embodiments, the flexibility of the substrate, the flexibility of the flexible backing, the adhesive strength of the first layer of adhesive, the affixation strength of the cord, and the pull strength of the cord are all within respective ranges such that when the first layer of adhesive has been adhered to the subject's body, and the first portion of the first thread has subsequently been moved in a direction that is parallel to the front face of the flexible backing, thereby detaching a portion of the flexible backing from the subject's body, the flexible backing can be peeled away from the subject's body by pulling on the cord.

In some embodiments of the third apparatus, each second portion of the first thread is at least 10 cm long.

Some embodiments of the third apparatus further comprise a second thread having (a) a first portion positioned in front of the flexible backing such that when the first layer of adhesive is adhered to the subject's body, the first portion of the second thread will be disposed between the flexible backing and the subject's body and (b) at least one second portion that extends beyond the perimeter of the flexible backing. In these embodiments, the first and second portions of the second thread are positioned with respect to the flexible backing so that pulling the at least one second portion of the second thread in a direction that is parallel to the front face of the flexible backing will cause the first portion of the second thread to move in a direction that is parallel to the front face of the flexible backing, thereby detaching at least a portion of the flexible backing from the subject's body.

Optionally, in the embodiments described in the previous paragraph, the first portion of the first thread is positioned so that an area bounded by the first portion of the first thread and the perimeter of the flexible backing is less than 10% of the total area, and the first portion of the second thread is positioned so that an area bounded by the first portion of the second thread and the perimeter of the flexible backing is less than 10% of the total area.

Optionally, in the embodiments described in the previous paragraph, the flexible backing has a first corner and a second corner, the area bounded by the first portion of the first thread and the perimeter of the flexible backing is located at the first corner, and the area bounded by the first portion of the second thread and the perimeter of the flexible backing is located at the second corner.

Some embodiments of the third apparatus further comprise a sheet of a polymer material with a dielectric constant of at least 10 disposed in front of the at least one conductive pad, in electrical contact with the at least one conductive pad.

Some embodiments of the third apparatus further comprise a sheet of graphite disposed in front of the at least one conductive pad, in electrical contact with the at least one conductive pad, and the sheet of graphite has a front face and a rear face. Optionally, these embodiments can further comprise a layer of conductive adhesive disposed between the at least one conductive pad and the rear face of the sheet of graphite, and a layer of conductive adhesive disposed on the front face of the sheet of graphite.

Some embodiments of the third apparatus further comprise a cable that is mounted to the substrate, and the cable includes at least one electrical conductor that is electrically connected to the at least one conductive pad.

In some embodiments of the third apparatus, the first thread comprises at least one of nylon and expanded polytetrafluoroethylene.

Another aspect of the invention is directed to a fourth apparatus for applying an electrical signal to a subject's body. The fourth apparatus comprises a flexible sheet of graphite, a first layer of conductive adhesive, and a first thread. The flexible sheet of graphite has a front face, a rear face, a total area, and an outer perimeter. The first layer of conductive adhesive is disposed on the front face of the sheet of graphite, and the first layer of conductive adhesive is biocompatible and is configured to adhere to the subject's body. The first thread has (a) a first portion positioned in front of the sheet of graphite such that when the first layer of conductive adhesive is adhered to the subject's body, the first portion of the first thread will be disposed between the sheet of graphite and the subject's body and (b) at least one second portion that extends beyond the perimeter of the sheet of graphite. The first and second portions of the first thread are positioned with respect to the sheet of graphite so that pulling the at least one second portion of the first thread in a direction that is parallel to the front face of the sheet of graphite will cause the first portion of the first thread to move in a direction that is parallel to the front face of the sheet of graphite, thereby detaching at least a portion of the sheet of graphite from the subject's body.

In some embodiments of the fourth apparatus, the first portion of the first thread is positioned so that an area bounded by the first portion of the first thread and the perimeter of the sheet of graphite is less than 10% of the total area.

In some embodiments of the fourth apparatus, the first portion of the first thread is positioned so that an area bounded by the first portion of the first thread and the perimeter of the sheet of graphite is less than 10% of the total area, and the sheet of graphite has a first corner, and the area bounded by the first portion of the first thread and the perimeter of the sheet of graphite is located at the first corner.

Optionally, the embodiments described in the previous paragraph further comprise a cord that is affixed to the sheet of graphite at a position that is less than 3 cm away from the first corner. In these embodiments, the flexibility of the sheet of graphite, the adhesive strength of the first layer of conductive adhesive, the affixation strength of the cord, and the pull strength of the cord are all within respective ranges such that when the first layer of conductive adhesive has been adhered to the subject's body, and the first portion of the first thread has subsequently been moved in a direction that is parallel to the front face of the sheet of graphite, thereby detaching a portion of the sheet of graphite from the subject's body, the sheet of graphite can be peeled away from the subject's body by pulling on the cord.

In some embodiments of the fourth apparatus, each second portion of the first thread is at least 10 cm long.

Some embodiments of the fourth apparatus further comprise a second thread having (a) a first portion positioned in front of the sheet of graphite such that when the first layer of conductive adhesive is adhered to the subject's body, the first portion of the second thread will be disposed between the sheet of graphite and the subject's body and (b) at least one second portion that extends beyond the perimeter of the sheet of graphite. In these embodiments, the first and second portions of the second thread are positioned with respect to the sheet of graphite so that pulling the at least one second portion of the second thread in a direction that is parallel to the front face of the sheet of graphite will cause the first portion of the second thread to move in a direction that is parallel to the front face of the sheet of graphite, thereby detaching at least a portion of the sheet of graphite from the subject's body.

Optionally, in the embodiments described in the previous paragraph, the first portion of the first thread is positioned so that an area bounded by the first portion of the first thread and the perimeter of the sheet of graphite is less than 10% of the total area, and the first portion of the second thread is positioned so that an area bounded by the first portion of the second thread and the perimeter of the sheet of graphite is less than 10% of the total area.

Optionally, in the embodiments described in the previous paragraph, the sheet of graphite has a first corner and a second corner, the area bounded by the first portion of the first thread and the perimeter of the sheet of graphite is located at the first corner, and the area bounded by the first portion of the second thread and the perimeter of the sheet of graphite is located at the second corner.

In some embodiments of the fourth apparatus, the first thread comprises at least one of nylon and expanded polytetrafluoroethylene.

### BRIEF DESCRIPTION OF THE DRAWINGS

FIG. 1 depicts an electrode assembly that can be peeled away from the subject's body by pulling on a cord or cords.

FIG. 2 depicts how the cords are used to peel the FIG. 1 electrode assembly away from the subject's body.

FIG. 3 depicts an electrode assembly that can be peeled away from the subject's body by pulling on a cable.

FIG. 4 depicts an electrode assembly that can be peeled away from the subject's body by pulling on a cable that is attached to a cord.

FIG. 5 depicts a situation that should be avoided for the FIG. 4 electrode assembly.

FIGS. 6 and 7 depict a two-part electrode assembly in which the front part can be peeled away from the subject's body by pulling on a cord, in conjoined and separated states, respectively.

FIG. 8 depicts one embodiment of a self-adhesive electrode assembly in which the adhesive bond between the electrode assembly and the subject's body is broken by sliding a thread in front of the electrode assembly.

FIG. 9 depicts a front view of the FIG. 8 electrode assembly.

FIGS. 10A and 10B depict another embodiment of a self-adhesive electrode assembly in which the adhesive bond between the electrode assembly and the subject's body is broken by sliding a thread in front of the electrode assembly, at two different stages in time.

FIGS. 11 and 12 respectively depict rear-perspective and front views of the front portion of an electrode assembly in which the adhesive bond between the front portion and the subject's body is broken by sliding a thread in front of the front portion.

Various embodiments are described in detail below with reference to the accompanying drawings, wherein like reference numerals represent like elements.

### DESCRIPTION OF THE PREFERRED EMBODIMENTS

This application is organized in two sections. The embodiments described in Section I rely on a cable or a cord that is affixed to each electrode assembly near the outer perimeter (e.g., near a corner) of the electrode assembly. After the electrode assembly has been adhered to the subject's body, the electrode assembly can be peeled away from the subject's body by pulling the cable or cord. The embodiments described in Section II rely on one or more threads that are configured to slide between the front face of the electrode assembly and the subject's body, in order to break the adhesive grip that holds the electrode assembly to the subject's body.

### Section I

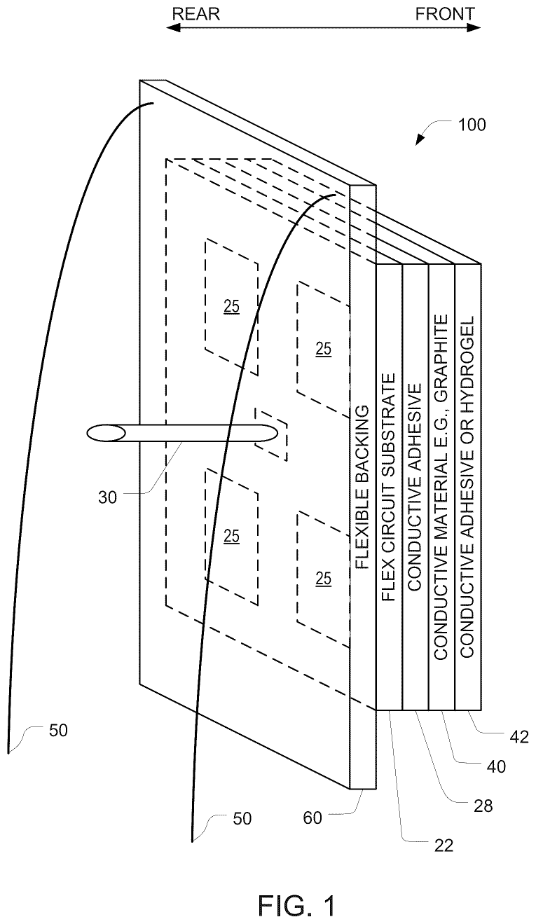

FIG. 1 depicts one embodiment of an electrode assembly that can be peeled away from the subject's body by pulling on a cable or cord. More specifically, FIG. 1 depicts an electrode assembly 100 that has a flex-circuit construction and can be peeled away from the subject's body by pulling on a cord 50.

The FIG. 1 embodiment is based on a flex circuit that has a flexible substrate 22 and at least one conductive pad 25 disposed on a front face of the flexible substrate. The flexible substrate and the at least one conductive pad can be implemented using any conventional flex circuit technology. For example, the flexible substrate 22 could be made from polyimide or another suitable insulating material, and the at least one conductive pad 25 can be made of copper or another metal. Additional details of how to implement an electrode assembly for applying TTFields to a subject's body using a flex circuit based architecture can be found in US Pub. 2021/0402179, which is incorporated herein by reference in its entirety.

A flexible backing 60 is affixed to the rear face of the flexible substrate 22. The affixation between the rear face of the flexible substrate 22 and the flexible backing 60 can be direct (e.g., using an adhesive). But in alternative embodiments, the affixation between the rear face of the flexible substrate 22 and the flexible backing 60 can be implemented via one or more intervening layers (not shown). The function of the flexible backing 60 is to support the flexible substrate and the rest of the electrode assembly 100, and the flexible backing 60 can be made from a cloth or foam material similar to corresponding materials that are used in self-adhesive bandages. Optionally, peripheral portions of the front face of the flexible backing 60 can be coated with an adhesive that is designed to adhere to skin, e.g., with an adhesive that is similar to adhesives used in self-adhesive bandages. This adhesive helps hold the electrode assembly 100 against the subject's body. In some embodiments, the entirety of the flexible backing 60 (or only the peripheral portions of the flexible backing 60) is made from a suitable foam material such as LDPE foam, silicone foam, polyurethane foam or ethylene-vinyl acetate (EVA) foam. In these embodiments, the foam is coated with a suitable adhesive that is designed to adhere to skin and to be removable (including but not limited to the adhesive that is used in the 3M™ TEGADERM™ high performance foam adhesive dressing).

In the embodiment depicted in FIG. 1, a flexible sheet of conductive material 40 (e.g., graphite) is positioned in front of the conductive pads 25 of the flex circuit, and this sheet of conductive material 40 is maintained in electrical contact with the conductive pads 25 by a layer of conductive adhesive 28. And the front face of the flexible sheet of conductive material 40 is coated with a layer 42 of conductive adhesive or conductive gel that is biocompatible and is configured to adhere to the subject's body.

Examples of suitable materials for flexible sheet of conductive material 40 include, but are not limited to, synthetic graphite, pyrolytic graphite (including, but not limited to, Pyrolytic Graphite Sheet (PGS), available from Panasonic Industry, Kadoma, Osaka, Japan), graphitized polymer film (e.g., graphitized polyimide film, including, but not limited to, that supplied by Kaneka Corp., Moka, Tochigi, Japan), or graphite foil made from compressed high purity exfoliated mineral graphite (including, but not limited to, that supplied by MinGraph® 2010A Flexible Graphite, available from Mineral Seal Corp., Tucson, Arizona, USA). In alternative embodiments, instead of using a layer of graphite, a layer of another anisotropic material or another flexible conductive material may be used.

Examples of suitable materials for the layer 42 of conductive adhesive include, but are not limited to, the OMNI-WAVE™ adhesive compositions manufactured and sold by FLEXCON® (Spencer, MA, USA), such as the developmental product FLX068983 FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M) may also be used. In other embodiments, a layer of conductive gel (e.g., conductive hydrogel) is used instead of the layer of conductive adhesive. In these embodiments, the level of adhesion between the layer 42 and the subject's body will be relatively weak, and will be based on the tackiness of the conductive gel.

Preferably, the adhesive strength of the layer 42 of conductive adhesive (or conductive gel) and the adhesive strength of the peripheral portions of the flexible backing 60 (when present) are collectively sufficient so that the electrode assembly 100 will remain attached to the subject's skin (and will not be dislodged) during ordinary daily activities (e.g., getting dressed or undressed). On the other hand, the adhesive strengths of the layer 42 of conductive adhesive (or conductive gel) and the peripheral portions of the flexible backing 60 (when present) should be low enough so that when the electrode assembly has been adhered to the subject's body, the flexible backing, the at least one conductive pad, and the flexible substrate can all be peeled away from the subject's body by pulling on the cord 50.

Thus, in this embodiment, a first layer 42 of conductive adhesive or conductive gel is disposed in front of the at least one conductive pad. In addition, the flexible sheet of conductive material 40 is positioned between the at least one conductive pad 25 and the first layer 42 of conductive adhesive or conductive gel; the flexible sheet of conductive material 40 is disposed in electrical contact with both the at least one conductive pad 25 and the first layer 42 of conductive adhesive or conductive gel; and the second layer of conductive adhesive 28 is disposed between the at least one conductive pad 25 and the rear face of the sheet of conductive material 40. Note however, that the flexible sheet of conductive material 40 and/or the second layer of conductive adhesive 28 can be omitted in alternative embodiments (not shown).

In the embodiment depicted in FIG. 1, two cords 50 are affixed to the flexible backing at respective corner positions, each of which is less than 3 cm away (e.g., <2 cm, <1.5 cm, or <1 cm) from a respective corner of the flexible backing 60. But in alternative embodiments (not shown), only a single cord 50 can be provided, or more than two cords 50 can be provided. Note also that instead of affixing the cord(s) to corner positions, the cord(s) can be affixed to the flexible backing at respective positions, each of which is less than 3 cm (e.g., <2 cm, <1.5 cm, or <1 cm) away from the outer perimeter of the flexible backing 60. The pull strength of the cords 50 should be sufficient such that when the electrode assembly 100 has been adhered to the subject's body, the entire electrode assembly 100 (including the flexible backing 60, the at least one conductive pad 25, and the flexible substrate 22) can all be peeled away from the subject's body by pulling on the cord(s).

Examples of suitable materials for the cords 50 include but are not limited to natural fibers (e.g., cotton, linen, etc.) and synthetic fibers (e.g., nylon, polyester, Dyneema®, etc.). In some embodiments, each of the cords 50 is at least 10 cm long.

In the embodiment depicted in FIG. 1, each of the cords 50 is affixed to the flexible backing 60. But in alternative embodiments (not shown), one or more of the cords 50 can be affixed to the flexible substrate 22 instead of to the flexible backing 60. In still other alternative embodiments (not shown), one or more of the cords 50 can be affixed to both the flexible backing 60 and the flexible substrate 22. Regardless of whether the cords 50 are affixed to the flexible backing 60 alone, to the flexible substrate 22 alone, or to both the flexible backing 60 and the flexible substrate 22, the cords 50 should be affixed with sufficient affixation strength such that when the electrode assembly 100 has been adhered to the subject's body, the entire electrode assembly 100 (including the flexible backing 60, the at least one conductive pad 25, and the flexible substrate 22) can all be peeled away from the subject's body by pulling on the cord(s) 50.

The electrode assembly 100 also has a cable 30 that is mounted to the substrate 22 in a manner that electrically connects at least one electrical conductor of the cable to the at least one conductive pad 25. This cable 30 is used to apply electrical signals to the at least one conductive pad 25. When two electrode assemblies 100 are positioned on the subject's skin on opposite sides of a region of interest, and an AC voltage is applied between those two electrode assemblies, an AC current is coupled into the subject's body, which generates TTFields in the region of interest. Note that in the embodiment depicted in FIG. 1, none of the cords 50 are electrically connected to the at least one conductive pad 25.

It is important to note that the thicknesses of the various layers of material depicted in all the figures are not drawn to scale, and in practice all of the layers will be much thinner than they appear to be in the figures. For example, all of the layers of material depicted in FIG. 1 will typically be less than 1 mm thick, and most of the layers will be less than 0.1 mm thick. Moreover, all of the layers should be sufficiently flexible so that the entire electrode assembly 100 can be peeled away from the subject's body. In particular, the substrate 22 and the flexible backing 60 should both be flexible enough so that when the electrode assembly 100 has been adhered to the subject's body it can be peeled away from the subject's body by pulling on the cord 50. In those embodiments that include additional layers (e.g., the layer of conductive material 40 and the second layer of conductive adhesive 28 depicted in FIG. 1), those layers should also be flexible enough so as not to interfere with the peeling away of the electrode assembly 100 from the subject's body when the cord 50 is pulled.

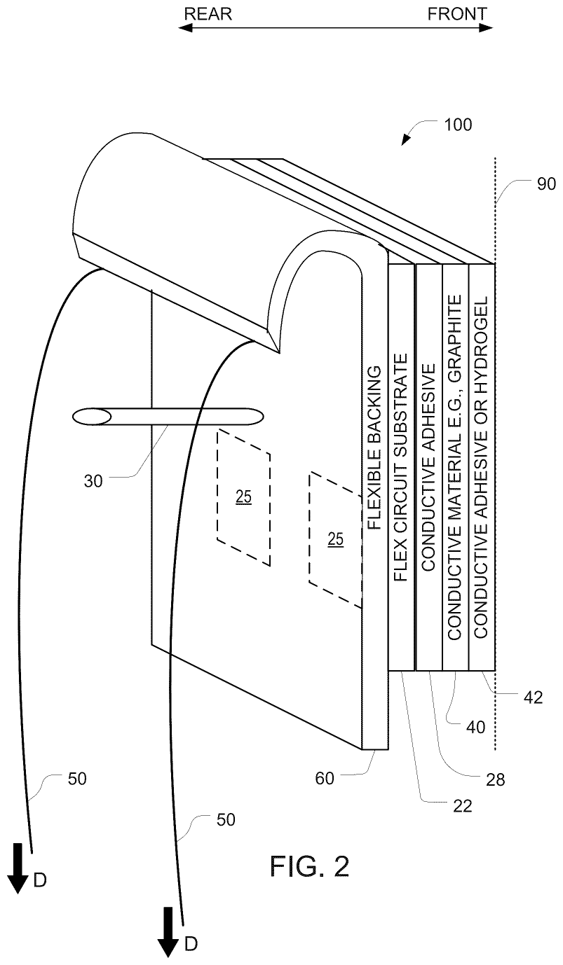

Turning now to FIG. 2, the cords 50 are used to peel the electrode assembly 100 away from the subject's body (at any time subsequent to the electrode assembly's affixation to the subject's body). Thus, in the example depicted in FIG. 2, in which the cords 50 are affixed to the flexible backing 60, if the cords 50 are pulled in the direction D, the flexible backing 60 will turn back on itself and begin to peel away from the subject's body, as depicted in FIG. 2. By continuing to pull on the cords 50 in the direction D, all the remaining layers positioned in front of the flexible backing 60 (i.e., the flexible substrate 22, the conductive adhesive 28, the layer of conductive material 40, and the layer 42 of conductive adhesive or conductive gel will all bend toward the left of the FIG. 2, so that the entire electrode assembly 100 turns back on itself and peels away from the subject's body.

To remove the electrode assembly 100 from the surface of a subject's body (e.g., the subject's skin), it is best to pull the cord 50 in a direction that deviates by <30° (e.g., <20°, <10°, <5°or 2°) from parallel to the surface of the subject's body, so that a first portion of the electrode assembly 100 bends back on itself. (The dotted line 90 in FIG. 2 represents the surface of the subject's body.) When the cord 50 is attached to a first corner of the electrode assembly 100 and the first cord is pulled in the direction indicated above, the electrode assembly 100 will bend back on itself beginning at the first corner and continuing until the electrode assembly 100 has peeled away from the surface of the subject's body. During an initial phase of the peeling-away motion, the first corner and a centroid of the flexible patch will get closer to each other.

In those embodiments that include a second cord 50 (e.g., the embodiment depicted in FIG. 2), when the first and second cords 50 are attached to first and second corners of the electrode assembly 100, respectively, and the cords are pulled in the direction indicated above, the electrode assembly 100 will bend back on itself beginning at both corners and continuing until the electrode assembly 100 has peeled away from the surface of the subject's body. During an initial phase of the peeling-away motion, the first corner and a centroid of the flexible patch will get closer to each other, and the second corner and the centroid of the flexible patch will also get closer to each other.

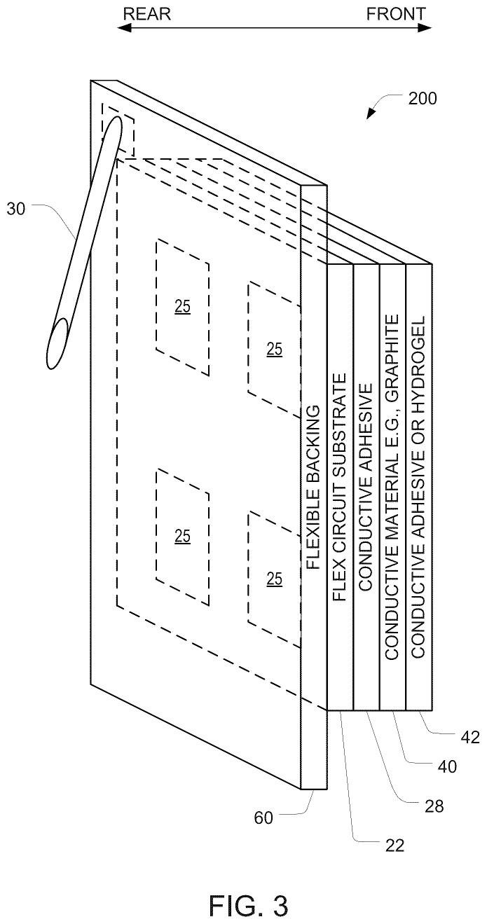

FIG. 3 depicts another embodiment of an electrode assembly that can be peeled away from the subject's body by pulling on a cable or cord. More specifically, FIG. 3 depicts an electrode assembly 200 that has a flex-circuit construction and can be peeled away from the subject's body by pulling on a cable 30.

The FIG. 3 embodiment is similar to the FIG. 1 embodiment, with similar reference numbers denoting similar items, except that cable 30 in the FIG. 3 embodiment does double duty. More specifically, the cable 30 in the FIG. 3 embodiment is used both to apply electrical signals to the at least one conductive pad 25, and also to peel the electrode assembly 200 away from the subject's body.

In this FIG. 3 embodiment, the adhesive strengths of the layer 42 of conductive adhesive (or conductive gel) and the peripheral portions of the flexible backing 60 (when present) should be low enough so that when the electrode assembly 200 has been adhered to the subject's body, the flexible backing, the at least one conductive pad, and the flexible substrate can all be peeled away from the subject's body by pulling on the cable 30.

In the embodiment depicted in FIG. 3, a cable 30 is affixed to the flexible backing at a corner position that is less than 3 cm away (e.g., <2 cm, <1.5 cm, or <1 cm) from a corner of the flexible backing 60. But in alternative embodiments (not shown), instead of affixing the cable 30 to a corner position, the cable 30 can be affixed to the flexible backing at a non-corner position that is less than 3 cm (e.g., <2 cm, <1.5 cm, or <1 cm) away from the outer perimeter of the flexible backing 60. The pull strength of the cable 30 should be sufficient such that when the electrode assembly 200 has been adhered to the subject's body, the entire electrode assembly 200 (including the flexible backing 60, the at least one conductive pad 25, and the flexible substrate 22) can all be peeled away from the subject's body by pulling on the cable 30.

In the embodiment depicted in FIG. 3, the cable 30 is affixed to the flexible backing 60. But in alternative embodiments (not shown), the cable 30 can be affixed to the flexible substrate 22 instead of to the flexible backing 60. In still other alternative embodiments (not shown), the cable 30 can be affixed to both the flexible backing 60 and the flexible substrate 22. Regardless of whether the cable 30 is affixed to the flexible backing 60 alone, to the flexible substrate 22 alone, or to both the flexible backing 60 and the flexible substrate 22, the cable 30 should be affixed with sufficient affixation strength such that when the electrode assembly 200 has been adhered to the subject's body, the entire electrode assembly 200 (including the flexible backing 60, the at least one conductive pad 25, and the flexible substrate 22) can all be peeled away from the subject's body by pulling on the cable 30.

In addition to being affixed to at least one of the flexible backing 60 and the substrate 22, the cable 30 is also mounted to the substrate 22 in a manner that electrically connects at least one electrical conductor of the cable to the at least one conductive pad 25. The cable 30 is used to apply electrical signals to the at least one conductive pad 25. When two electrode assemblies 200 are positioned on the subject's skin on opposite sides of a region of interest, and an AC voltage is applied between those two electrode assemblies, an AC current is coupled into the subject's body, which generates TTFields in the region of interest.

The peeling away of the electrode assembly 200 from the subject's body by pulling on the cable 30 is similar to the way the electrode assembly 100 is pulled away from the subject's body by pulling on the cord 50 in the FIG. 1 embodiment described above.

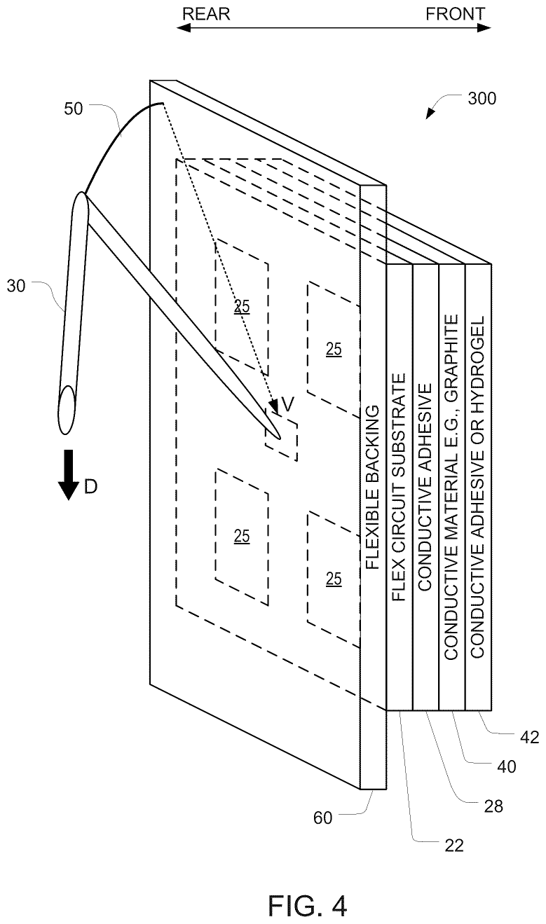

FIG. 4 depicts another embodiment of an electrode assembly that can be peeled away from the subject's body by pulling on a cable or cord. More specifically, FIG. 4 depicts an electrode assembly 300 that has a flex-circuit construction and can be peeled away from the subject's body by pulling on a cable 30 that is attached to a cord 50.

The FIG. 4 embodiment is similar to the FIG. 1 embodiment, with similar reference numbers denoting similar items, except that cable 30 in the FIG. 4 embodiment is used both to apply electrical signals to the at least one conductive pad 25, and also to peel the electrode assembly 300 away from the subject's body by exerting a pull force on the cord 50.

In the FIG. 4 embodiment,, the adhesive strengths of the layer 42 of conductive adhesive (or conductive gel) and the peripheral portions of the flexible backing 60 (when present) should be low enough so that when the electrode assembly 300 has been adhered to the subject's body, the flexible backing, the at least one conductive pad, and the flexible substrate can all be peeled away from the subject's body by pulling on the cable 30, which exerts a pull force on the cord 50.

In the embodiment depicted in FIG. 4, a cord 50 is affixed to the flexible backing at a corner position that is less than 3 cm away (e.g., <2 cm, <1.5 cm, or <1 cm) from a corner of the flexible backing 60. Note also that instead of affixing the cord 50 to a corner position, the cord 50 can be affixed to the flexible backing at a non-corner position that is less than 3 cm (e.g., <2 cm, <1.5 cm, or <1 cm) away from the outer perimeter of the flexible backing 60. The same materials described above for the cord 50 in the FIG. 1 embodiment can be used for the cord 50 in the FIG. 4 embodiment. In the embodiment depicted in FIG. 4, the cord 50 is affixed to the flexible backing 60. But in alternative embodiments (not shown), the cord 50 can be affixed to the flexible substrate 22 instead of to the flexible backing 60. In still other alternative embodiments (not shown), the cord 50 can be affixed to both the flexible backing 60 and the flexible substrate 22.

The electrode assembly 300 also has a cable 30 that is mounted to the substrate 22 in a manner that electrically connects at least one electrical conductor of the cable to the at least one conductive pad 25. This cable 30 is used to apply electrical signals to the at least one conductive pad 25. When two electrode assemblies 300 are positioned on the subject's skin on opposite sides of a region of interest, and an AC voltage is applied between those two electrode assemblies, an AC current is coupled into the subject's body, which generates TTFields in the region of interest.

The cable 30 in this FIG. 4 embodiment is attached to the cord 50 less than 5 cm away from a point at which the cord 50 is affixed to at least one of the substrate 22 and the flexible backing 60. The cable 30 and the cord 50 are configured so that pull forces applied to the cable 30 in a direction that is parallel to a vector V that runs from (a) the point at which the cord 50 is affixed to at least one of the substrate 22 and the flexible backing 60 to (b) a centroid of the flexible backing 60 are transmitted to the cord 50 via the cable-to-cord attachment.

Regardless of whether the cord 50 is affixed to the flexible backing 60 alone, to the flexible substrate 22 alone, or to both the flexible backing 60 and the flexible substrate 22, the cord 50 should be affixed with sufficient affixation strength such that when the electrode assembly 300 has been adhered to the subject's body, the entire electrode assembly 300 (including the flexible backing 60, the at least one conductive pad 25, and the flexible substrate 22) can all be peeled away from the subject's body by pulling on the first cord.

The pull strength of the cord 50, the pull strength of the cable 30, and the strength of attachment between the cord 50 and the cable 30 should all be sufficient such that when the electrode assembly 300 has been adhered to the subject's body, the entire electrode assembly 300 (including the flexible backing 60, the at least one conductive pad 25, and the flexible substrate 22) can all be peeled away from the subject's body by pulling on the cable 30, which transmits the pulling force to the cord 50 via the cable-to-cord attachment, which peels the electrode assembly 300 away from the subject's body.

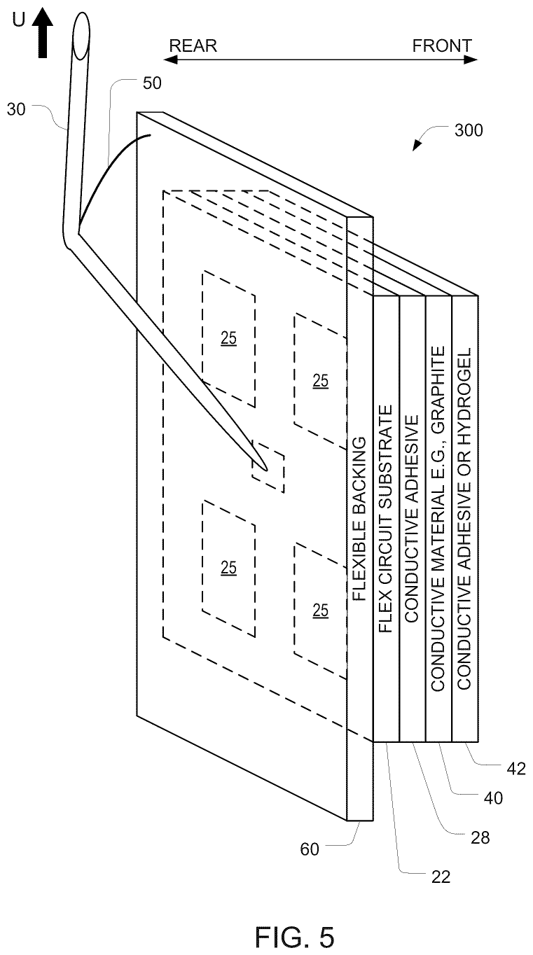

In the FIG. 4 embodiment, it is best to pull the cable 30 in the direction D so that the cable 30 will transmit the pulling force to the cord 50, which will peel the electrode assembly 300 away from the subject's body in a manner that is similar to the peeling depicted in FIG. 2. This is because pulling the cable 30 in the opposite direction U (as depicted in FIG. 5) will not cause the electrode assembly 300 to fold back on itself and peel away from the subject's body. Pulling the cable 30 in the direction U depicted in FIG. 5 should therefore be avoided.

While the embodiments discussed above in connection with FIGS. 1-5 are described in the context of a flex circuit based architecture, alternative architectures in which at least one conductive pad is supported by a flexible substrate can be used instead of the flex circuit based architecture. One example of such an architecture is to use a flexible substrate to support a plurality of ceramic discs with a metallization layer disposed on the rear surface of each ceramic disc, and a layer of hydrogel disposed on the front surface of each ceramic disc. A flexible backing is affixed to the rear face of the flexible substrate and supports both the flexible substrate and the ceramic discs. When a cord is attached to the flexible backing and/or the flexible substrate less than 3 cm away from the outer perimeter of the flexible backing, the cord can be used to peel the electrode assembly away from the subject body in a manner similar to the manner described above in connection with FIG. 2.

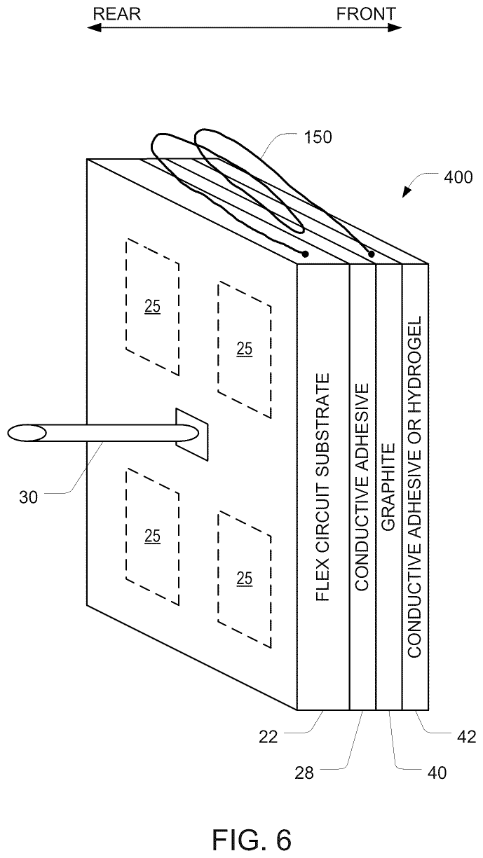

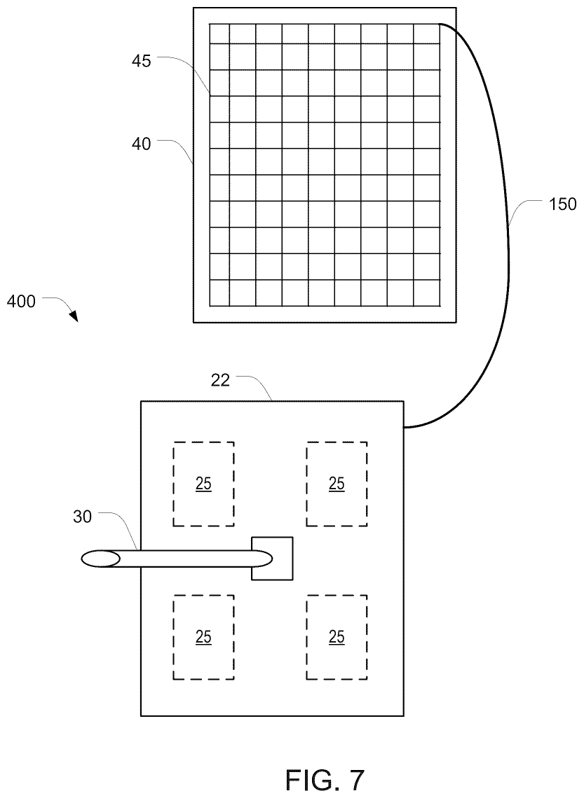

FIGS. 6 and 7 depict a two-part electrode assembly 400 in which the front part can be peeled away from the subject's body by pulling on cord 150. More specifically, FIG. 6 depicts a side view of this electrode assembly 400 when both parts of the electrode assembly are pressed together (in which case they can be used to apply TTFields to a subject's body), and FIG. 7 depicts this electrode assembly 400 when the two parts of the electrode assembly have been separated.

The rear portion of the two-part electrode assembly 400 is a flex circuit that has a flexible substrate 22 and at least one conductive pad 25 disposed on a front face of the flexible substrate. The flexible substrate 22 and the at least one conductive pad 25 can be configured and arranged as the correspondingly-numbered components described above in connection with FIGS. 1-2. Note that while the rear portion of the two-part electrode assembly 400 depicted in FIGS. 6-7 is constructed using a flex circuit, alternative architectures for the rear portion (e.g., electrode discs that are hardwired to a connector) may also be used.

The front portion of the two-part electrode assembly 400 is a flexible sheet of graphite 40 that has a front face, a rear face, and an outer perimeter. A first layer of conductive adhesive 42 is disposed on the front face of the sheet of graphite, and this first layer of conductive adhesive is biocompatible and is configured to adhere to the subject's body. Examples of suitable materials for flexible sheet of graphite 40 include, but are not limited to, synthetic graphite, pyrolytic graphite, graphitized polymer film, or graphite foil, similar to the corresponding items described above in connection with FIGS. 1-2. Examples of suitable materials for the layer 42 of conductive adhesive include any of the conductive adhesives described above in connection with FIGS. 1-2. Optionally, a grid of reinforcing threads 45 can be bonded to the sheet of graphite 40.

The front and rear portions of the two-part electrode assembly 400 depicted in FIG. 6 are held together by a layer of conductive adhesive 28. And a cable 30 is mounted to the substrate 22 (on the rear portion of the assembly 400) in a manner that electrically connects at least one electrical conductor of the cable to the at least one conductive pad 25. This cable 30 is used to apply electrical signals to the at least one conductive pad 25.

One end of a cord 150 is affixed to the sheet of graphite 40 at a position that is less than 3 cm away from the outer perimeter of the sheet of graphite. The affixation of the cord 150 to the sheet of graphite 40 may be a direct affixation. Alternatively, the affixation of the cord 150 to the sheet of graphite 40 may be indirect via one or more intervening components (e.g., by affixing the cord 150 to the grid of reinforcing threads 45 that are bonded to the sheet of graphite 40). The other end of the cord 150 is affixed to the rear portion of the two-part electrode assembly 400 (e.g., to the flexible substrate 22). The same materials described above for the cord 50 in the FIG. 1 embodiment can be used for the cord 150 in the FIG. 6 embodiment.

When two electrode assemblies 400 are positioned on the subject's skin on opposite sides of a region of interest (with the front and rear portions of the electrode assembly 400 contacting each other), and an AC voltage is applied between those two electrode assemblies via their respective cables 30, an AC current is coupled into the subject's body, which generates TTFields in the region of interest.

After being used to apply TTFields, the electrode assemblies 400 are removed from the subject body. This can be accomplished by first peeling the rear portion of the electrode assembly 400 away from the front portion of that assembly 400. This can be accomplished, for example, by pulling on the cable 30 until the rear portion of the electrode assembly 400 separates from the front portion. After the two portions of the electrode assembly 400 have separated, the two portions will be situated as depicted in FIG. 7, in which the front portion of the electrode assembly remains affixed to the subject's body, the rear portion of the electrode assembly is separated from the front portion, and the front and rear portions are connected by the cord 150.

In some preferred embodiments, the adhesive strength of the middle layer of conductive adhesive 28 is lower than the adhesive strength of the front layer of conductive adhesive 42. This will facilitate the separation of the rear portion of the electrode assembly 400 from the front portion when the cable 30 is pulled.

After the two portions of the electrode assembly 400 have separated (as depicted in FIG. 7), the front part of the electrode assembly 400 can be peeled away from the subject's body by pulling further on the cable 30 so that the rear part of the electrode assembly 400 moves away from the front part until the cord 150 becomes taut. Further pulling on the cable 30 will pull the cord 150, which will peel the sheet of graphite 40 away from the subject's body (in a manner similar to the way that the electrode assembly 100 is peeled away from the subject's body as described above in connection with FIG. 2). The flexibility of the sheet of graphite 40, the adhesive strength of the front layer of conductive adhesive 42, the affixation strength of the cord 150, and the pull strength of the cord 150 should all be within respective ranges that will facilitate such peeling.

In some embodiments, the cord 150 is affixed to the sheet of graphite 40 at a position that is less than 1.5 cm away from the outer perimeter of the sheet of graphite. In some embodiments, the cord 150 is affixed to the sheet of graphite at a position that is less than 3 cm (e.g., <2 cm, <1.5 cm, or <1 cm) away from a corner of the sheet of graphite. In some embodiments, the cord 150 is at least 10 cm long.

In the embodiment depicted in FIGS. 6 and 7, a single cord 150 runs between the front and rear portions of the electrode assembly 400, and that cord is affixed near a corner of the front portion of the electrode assembly. But in alternative embodiments, an additional cord (not shown) can be affixed near another corner of the front portion of the electrode assembly. In these alternative embodiments, each cord is affixed to the sheet of graphite at a position that is less than 3 cm away (e.g., <2 cm, <1.5 cm, or <1 cm) from a respective corner of the sheet of graphite. The flexibility of the sheet of graphite, the adhesive strength of the front layer of conductive adhesive, the affixation strength of both cords, and the pull strength of both cords should all be within respective ranges such that when the front layer of conductive adhesive has been adhered to the subject's body, the sheet of graphite can be peeled away from the subject's body by pulling on either cord.

### Section II

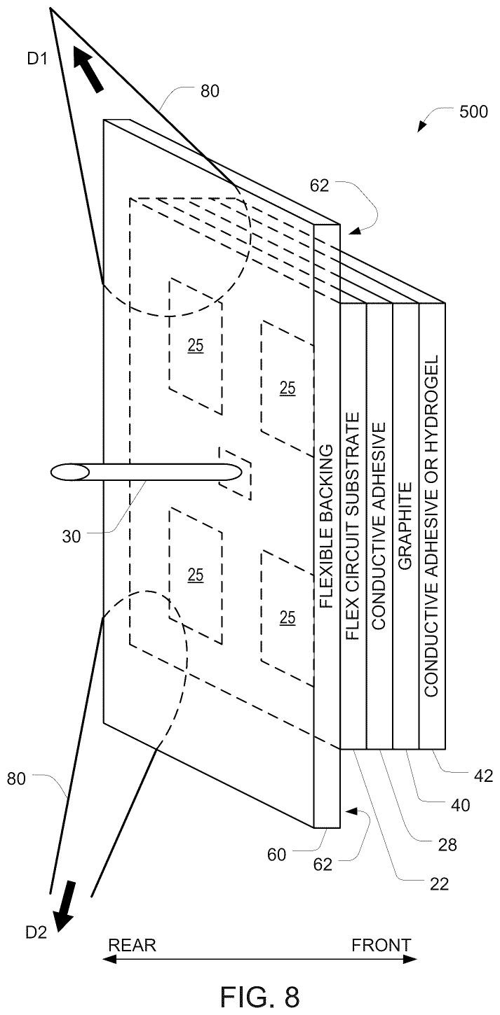

FIG. 8 depicts one embodiment of a self-adhesive electrode assembly 500 that can be easily removed from the subject's body. It includes one or more threads 80 that are configured to slide between the front face of the electrode assembly and the subject's body, in order to break the adhesive grip that holds the electrode assembly to the subject's body. Note that the thicknesses of the various layers of material depicted in all the figures are not drawn to scale, and in practice all of the layers will be much thinner than they appear to be in the figures. For example, all of the layers of material depicted in FIG. 8 will typically be less than 1 mm thick, and most of the layers will be less than 0.1 mm thick.

The FIG. 8 embodiment is based on a flex circuit that has a flexible substrate 22 and at least one conductive pad 25 disposed on a front face of the flexible substrate. The flexible substrate and the at least one conductive pad can be implemented using any conventional flex circuit technology, e.g., as described above in Section I.

A flexible backing 60 is affixed to the rear face of the flexible substrate 22. The affixation between the rear face of the flexible substrate 22 and the flexible backing 60 can be direct (e.g., using an adhesive). But in alternative embodiments, the affixation between the rear face of the flexible substrate 22 and the flexible backing 60 can be implemented via one or more intervening layers (not shown). The function of the flexible backing 60 is to support the flexible substrate and the rest of the electrode assembly 500, and the flexible backing 60 can be made from a cloth or foam material similar to corresponding materials that are used in self-adhesive bandages. Peripheral portions of the front face of the flexible backing 60 are coated with an adhesive 62 that is configured to adhere to the subject's skin, e.g., with an adhesive that is similar to adhesives used in self-adhesive bandages. This adhesive 62 holds the electrode assembly 500 against the subject's body. In some embodiments, the entirety of the flexible backing 60 (or only the peripheral portions of the flexible backing 60) is made from a suitable foam material such as LDPE foam, silicone foam, polyurethane foam or ethylene-vinyl acetate (EVA) foam. In these embodiments, the foam is coated with a suitable adhesive 62 that is designed to adhere to skin and to be removable (including but not limited to the adhesive that is used in the 3M™ TEGADERM™ high performance foam adhesive dressing).

In the embodiment depicted in FIG. 8, a flexible sheet of conductive material 40 (e.g., graphite) is positioned in front of the conductive pads 25 of the flex circuit, and this sheet of conductive material 40 is maintained in electrical contact with the conductive pads 25 by a layer of conductive adhesive 28. And the front face of the flexible sheet of conductive material 40 is coated with a layer 42 of conductive adhesive or conductive gel that is biocompatible and is configured to adhere to the subject's body.

Examples of suitable materials for the flexible sheet of conductive material 40 include, but are not limited to the materials described above in Section I.

Examples of suitable materials for the layer 42 of conductive adhesive include, but are not limited to the materials described above in Section I.

The same conductive adhesives described above in connection with the layer 42 can be used to implement the layer of conductive adhesive 28.

The electrode assembly 500 also has a cable 30 that is mounted to the substrate 22 in a manner that electrically connects at least one electrical conductor of the cable to the at least one conductive pad 25. This cable 30 is used to apply electrical signals to the at least one conductive pad 25. (Note that the cable 30 can be mounted directly to the substrate 22 or can be mounted to the substrate 22 via intervening components.) When two electrode assemblies 500 are positioned on the subject's skin on opposite sides of a region of interest, and an AC voltage is applied between those two electrode assemblies, an AC current is coupled into the subject's body, which generates TTFields in the region of interest.

The adhesive strength of the layer 42 of conductive adhesive (or conductive gel) and the adhesive strength of the adhesive 62 on the peripheral portions of the flexible backing 60 are preferably collectively sufficient so that the electrode assembly 500 will remain attached to the subject's skin (and will not be dislodged) during ordinary daily activities (e.g., getting dressed or undressed) for the duration of time (e.g., 10-14 days) that the electrode assembly remains on the subject's body.

Notably, the electrode assembly 500 includes at least one thread 80 that is configured to slide between the front face of the electrode assembly and the subject's body, in order to break the adhesive grip that holds the electrode assembly to the subject's body. This added feature helps facilitate removal of the electrode assembly 500 (which, in the absence of this feature, could be difficult to remove).

Each of the threads 80 has a first portion (depicted in dashed lines) positioned in front of the flexible backing 60 such that when the first layer of adhesive 62 is adhered to the subject's body, the first portion of the first thread will be disposed between the flexible backing and the subject's body. Each thread 80 also has at least one second portion (depicted in solid lines) that extends beyond the perimeter of the flexible backing. The first and second portions of each thread 80 are positioned with respect to the flexible backing 60 so that pulling the second portions of the first thread in a direction (e.g., D1 or D2) that is parallel to the front face of the flexible backing 60 will cause the first portion of the thread 80 to move in a direction that is parallel to the front face of the flexible backing 60, thereby breaking the adhesive grip that holds the electrode assembly 500 to the subject's body (and simultaneously detaching at least a portion of the flexible backing 60 from the subject's body). The threads 80 are preferably thin (to facilitate the parallel sliding to break the adhesive grip), and made of a relatively strong material (e.g., nylon, expanded polytetrafluoroethylene, etc.) to prevent breaking. In this regard, the thread 80 is similar to certain varieties of dental floss.

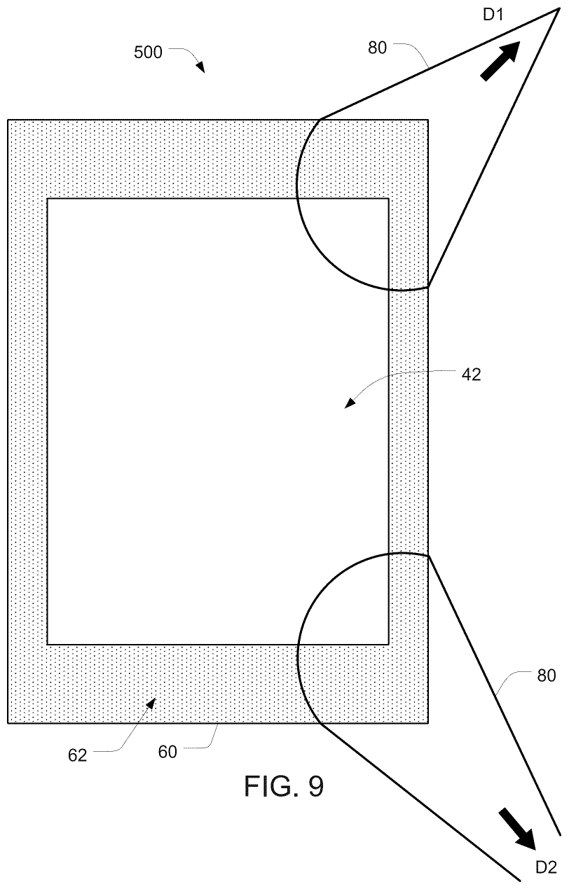

FIG. 9 depicts a front view of the electrode assembly 500, which clarifies that each of the threads 80 is positioned in front of the entire electrode assembly 500 (including but not limited to the layer 42 of conductive adhesive or hydrogel, the flexible backing 60, and the adhesive 62 disposed on the front face of the flexible backing 60).

Returning to FIG. 8, for each of the threads 80, the at least one second portion could be a single second portion configured in a closed loop (e.g., as shown in the top left of FIG. 8), in which case the closed loop is pulled in the direction D1 to break the adhesive grip that holds the electrode assembly 500 to the subject's body. Alternatively, the at least one second portion can be two distinct second portions (e.g., as shown on the bottom left of FIG. 8), in which case both second portions should be pulled in the direction D2 to break the adhesive grip that holds the electrode assembly 500 to the subject's body. In some embodiments, each second portion of the thread 80 is at least 10 cm long. In other embodiments, each second portion of the thread can be at least 2 cm, 5 cm, 8 cm, 15 cm, or 20 cm long (e.g., 2-20 cm, 5-20 cm, 8-20 cm, 15-30 cm, or 20-30 cm.)

For each of the threads 80, the first portion of the thread can be positioned so that an area bounded by the first portion of the thread and the perimeter of the flexible backing 60 is less than 20% of the total area (e.g., <5%, <8%, <10%, or <15% of the total area). Although this area can be located at a corner of the flexible backing 60 (as depicted in FIG. 8), the corner is not the only location for each of the threads 80 to be positioned. To the contrary, any given thread 80 can be positioned so that the area bounded by the first portion of the thread and the perimeter of the flexible backing 60 is located along a side of the flexible backing 60.

Advantageously, the thread(s) 80 facilitate easy removal of the electrode assembly 500 from the subject's body, because sliding the threads between the front face of the electrode assembly and the subject's skin breaks the adhesive grip that holds a portion of the electrode assembly (e.g., a corner) to the skin. And once the adhesive grip has been broken for this portion, it becomes much easier to peel the electrode assembly 500 away from the subject body (e.g., by grabbing the freed portion of the electrode assembly 500 between the thumb and forefinger, and pulling on the electrode assembly).

Note that while FIGS. 8-9 depict two threads 80, the number of threads 80 can vary (e.g., between 1 and 4).

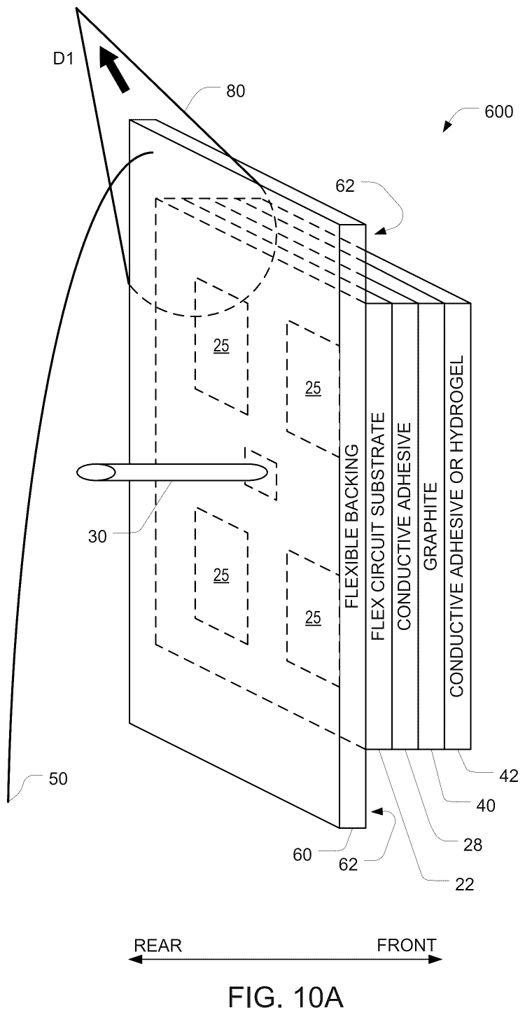

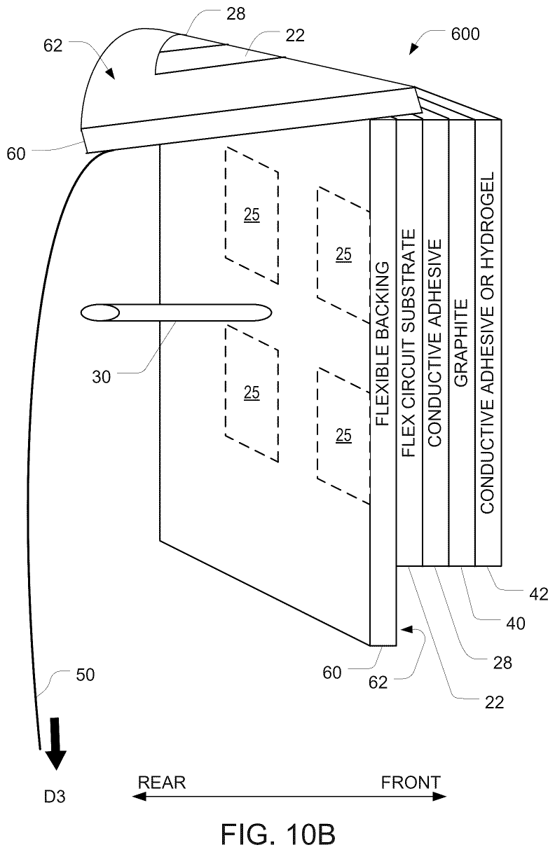

The electrode assembly 600 depicted in FIGS. 10A-B is similar to the electrode assembly 500 depicted in FIG. 8, except it has one additional component. More specifically, a cord 50 is affixed to the flexible backing 60 less than 3 cm away (e.g., <2 cm, <1.5 cm, or <1 cm) from a corner of the flexible backing 60. The pull strength of the cord 50 (as well as the affixation strength of the cord 50 to the flexible backing 60) should be sufficient such that when the electrode assembly 600 has been adhered to the subject's body, the entire electrode assembly 600 (including but not limited to the flexible backing 60) can be peeled away from the subject's body by pulling the cord 50 in the direction D3, as best seen in FIG. 10B. Examples of suitable materials for the cord 50 include but are not limited to natural fibers (e.g., cotton, linen, etc.) and synthetic fibers (e.g., nylon, polyester, Dyneema®, etc.). In some embodiments, the cord 50 is at least 10 cm long.

All of the layers of the FIGS. 10A-B electrode assembly 600 are preferably sufficiently flexible so the electrode assembly can be peeled away from the subject's body by pulling on the cord 50. In particular, the substrate 22 and the flexible backing 60 are preferably both flexible enough so that when the electrode assembly 600 has been adhered to the subject's body, it can be peeled away from the subject's body by pulling on the cord 50. In those embodiments that include additional layers (e.g., the layer of conductive material 40 and the second layer of conductive adhesive 28 depicted in FIGS. 10A-B), those layers are also preferably flexible enough so as not to interfere with the peeling away of the electrode assembly 600 from the subject's body when the cord 50 is pulled. In addition, the adhesive strength of the layer 42 of conductive adhesive (or conductive gel) and the adhesive strength of the adhesive 62 on the peripheral portions of the flexible backing 60 should be low enough so as not to interfere with the peeling away of the electrode assembly 600 from the subject's body when the cord 50 is pulled.

In the embodiment depicted in FIGS. 10A-B, the cord 50 is affixed to the flexible backing 60. But in alternative embodiments (not shown), the cord 50 can be affixed to the flexible substrate 22 instead of to the flexible backing 60. In still other alternative embodiments (not shown), the cord 50 can be affixed to both the flexible backing 60 and the flexible substrate 22. Regardless of whether the cord 50 is affixed to the flexible backing 60 alone, to the flexible substrate 22 alone, or to both the flexible backing 60 and the flexible substrate 22, the cord 50 should be affixed with sufficient affixation strength such that when the electrode assembly 600 has been adhered to the subject's body, the entire electrode assembly 600 can be peeled away from the subject's body by pulling on the cord.

Regardless of whether the cord 50 is affixed to the flexible backing 60 alone, to the flexible substrate 22 alone, or to both the flexible backing 60 and the flexible substrate 22, the best position to affix the cord 50 is at a location that is within the area bounded by the first portion of the thread 80 and the perimeter of the flexible backing 60, as best seen in FIG. 10A. Thus, in the FIG. 10A example, the cord 50 should be affixed at the top left of the flexible backing 60, above and to the left of the dashed semicircle (which represents the initial position of the thread 80). This is because moving the thread 80 in direction D1 will break the adhesive grip that holds the electrode assembly 500 to the subject's body (and simultaneously detach a portion of the flexible backing 60 from the subject's body). And after this portion of the flexible backing 60 has been detached, it will be much easier to use the cord 50 to peel the electrode assembly 600 away from the subject's body as depicted in FIG. 10B by pulling the cord 50 in the direction D3.

Note that in some embodiments, a sheet of polymer material (not shown) with a dielectric constant of at least 10 (e.g., >15 or >20) can be used in place of the sheet of graphite depicted in FIGS. 8-10. And in these embodiments, instead of using the conductive layers 28, 40, 42 to establish a conductive coupling between the at least one conductive pad 25 and the subject's body (as described above in connection with FIG. 8), the polymer material will establish a capacitive coupling between the at least one conductive pad 25 and the subject's body.

While the embodiments discussed above in connection with FIGS. 8-10 are described in the context of a flex circuit based architecture, alternative architectures in which at least one conductive pad is supported by a flexible substrate can be used instead of the flex circuit based architecture. One example of such an architecture is to use a flexible substrate to support a plurality of ceramic discs with a metallization layer disposed on the rear surface of each ceramic disc, and a layer of hydrogel disposed on the front surface of each ceramic disc. A flexible backing is affixed to the rear face of the flexible substrate and supports both the flexible substrate and the ceramic discs. At least one thread is configured to slide between the front face of the electrode assembly and the subject's body, in order to break the adhesive grip that holds the electrode assembly to the subject's body and facilitate removal of the electrode assembly, in a manner similar to the manner described above in connection with FIGS. 8-10.

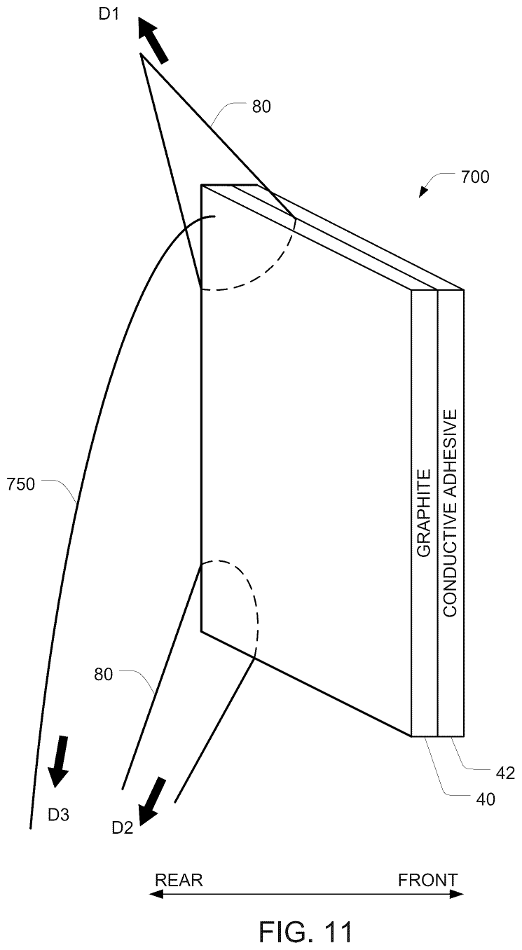

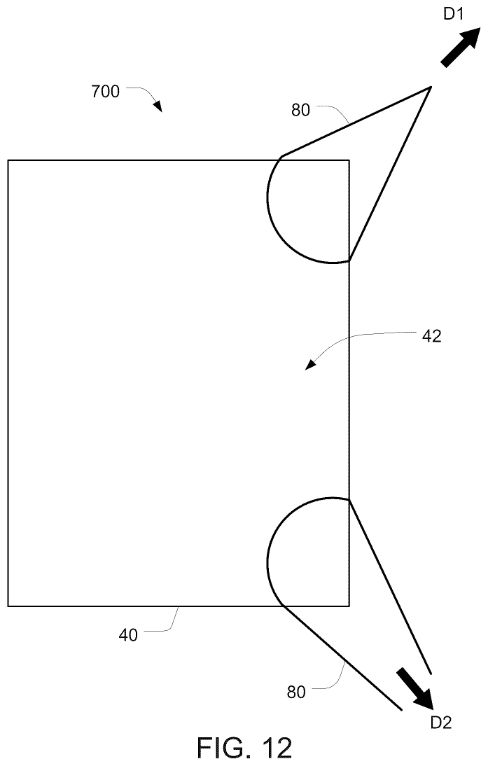

The electrode assemblies depicted in FIGS. 8 and 10A can be designed in two parts that can be either held together by the adhesive layer 28 or separated from each other. FIGS. 11 and 12 depict rear-perspective and front views of the self-adhesive front portion 700 a such a two-part electrode assembly that has been separated from the rear portion (i.e., separated from the rear layers depicted in FIG. 8).

The front portion 700 of the two-part electrode assembly is a flexible sheet of graphite 40 that has a front face, a rear face, a total area, and an outer perimeter. A first layer of conductive adhesive 42 is disposed on the front face of the sheet of graphite, and this first layer of conductive adhesive is biocompatible and is configured to adhere to the subject's body. The front portion 700 of the two-part electrode assembly is held to the rear portion of the two-part electrode assembly by a layer of conductive adhesive, which can be disposed either on the rear face of the sheet of graphite 40 or the front face of the rear portion of the two-part electrode assembly. This layer of conductive adhesive is similar to the layer of conductive adhesive 28 described above in connection with FIG. 8.

All of the graphite versions of the flexible sheet of conductive material 40 described above in connection with the FIG. 8-10 embodiments can be used in this FIGS. 11-12 embodiment. And all of the conductive adhesives described above in connection with the FIGS. 8-10 embodiments can be used for the layer 42 of conductive adhesive in this FIGS. 11-12 embodiment.

Similar to the electrode assembly 500 discussed above in connection with FIG. 8-10, the electrode assembly 700 also includes at least one thread 80 that is configured to slide between the front face of the electrode assembly 400 and the subject's body, in order to break the adhesive grip that holds the electrode assembly to the subject's body. This added feature helps facilitate removal of the electrode assembly.

Each of the threads 80 has a first portion (depicted in dashed lines in FIG. 11) positioned in front of the sheet of graphite 40 such that when the first layer of conductive adhesive 42 is adhered to the subject's body, the first portion of the thread 80 will be disposed between the sheet of graphite 40 and the subject's body. Each thread 80 also has at least one second portion that extends beyond the perimeter of the sheet of graphite 40. The first and second portions of the thread 80 are positioned with respect to the sheet of graphite 40 so that pulling the at least one second portion of the thread 80 in a direction that is parallel to the front face of the sheet of graphite 40 will cause the first portion of the thread 80 to move in a direction that is parallel to the front face of the sheet of graphite 40, thereby breaking the adhesive grip that holds the electrode assembly 700 to the subject's body (and simultaneously detaching a portion of the sheet of graphite 40 from the subject's body). The construction of the threads 80 in this FIGS. 11-12 embodiment is similar to the construction of the threads 80 discussed above in connection with FIGS. 8-10.

FIG. 12 depicts a front view of the electrode assembly 700, which clarifies that each of the threads 80 is positioned in front of the entire electrode assembly 700 (including the layer 42 of conductive adhesive and the sheet of graphite 40).

Each of threads 80 can be configured in a closed loop or with two distinct second portions, as described above in connection with FIGS. 8-10, and the lengths of each of the threads 80 as well as the total number of threads is also as described above in connection with FIGS. 8-10.

For each of the threads 80, the first portion of the thread can be positioned so that an area bounded by the first portion of the thread and the perimeter of the sheet of graphite 40 is less than 20% of the total area (e.g., <5%, <8%, <10%, or <15% of the total area). Although this area can be located at a corner of the sheet of graphite 40 (as depicted in FIG. 11), the corner is not the only location for each of the threads 80 to be positioned. To the contrary, any given thread 80 can be positioned so that the area bounded by the first portion of the thread and the perimeter of the sheet of graphite 40 is located along a side of the sheet of graphite 40.

Optionally, a cord 750 can be affixed to the sheet of graphite 40 at a position that is less than 3 cm (e.g., <2 cm, <1.5 cm, or <1 cm) away from one of the corners of the sheet of graphite, as depicted in FIG. 11. The affixation of the cord 750 to the sheet of graphite 40 may be a direct affixation. Alternatively, the affixation of the cord 750 to the sheet of graphite 40 may be indirect via one or more intervening components (e.g., by affixing the cord 750 to a grid of reinforcing threads (not shown) that are bonded to the sheet of graphite 40). The same materials and parameters described above for the cord 50 in the FIGS. 10A-B embodiment can be used for the cord 750 in the FIG. 11 embodiment.

If the first layer of conductive adhesive 42 has been adhered to the subject's body, and the first portion of the first thread 80 has subsequently been moved in a direction that is parallel to the front face of the sheet of graphite 40 (e.g., D1 or D2), a portion of the sheet of graphite 40 will become detached from the subject's body. The front portion 700 of the two-part electrode assembly (including the sheet of graphite) can then be peeled away from the subject's body by pulling the cord 750 in the direction D3. (This is similar to the way that the electrode assembly 600 is peeled away from the subject's body as described above in connection with FIGS. 10A-B). The flexibility of the sheet of graphite 40, the adhesive strength of the front layer of conductive adhesive 42, the affixation strength of the cord 750, and the pull strength of the cord 750 should all be within respective ranges that will facilitate such peeling.

Advantageously, the thread(s) 80 facilitate easy removal of the electrode assembly 700 from the subject's body, for the same reasons described above in connection with the FIG. 8-10 embodiments.

One aspect of this Section II is directed to a third apparatus for applying an electrical signal to a subject's body. The third apparatus comprises a flexible substrate, at least one conductive pad, a flexible backing, a first layer of adhesive, and a first thread. The flexible substrate has a front face and a rear face. The at least one conductive pad is disposed on the front face of the substrate. The flexible backing is affixed to the rear face of the flexible substrate, and the flexible backing has a total area and an outer perimeter. The first layer of adhesive is disposed on the front face of the flexible backing, and the first layer of adhesive is configured to adhere to the subject's body. The first thread has (a) a first portion positioned in front of the flexible backing such that when the first layer of adhesive is adhered to the subject's body, the first portion of the first thread will be disposed between the flexible backing and the subject's body and (b) at least one second portion that extends beyond the perimeter of the flexible backing. The first and second portions of the first thread are positioned with respect to the flexible backing so that pulling the at least one second portion of the first thread in a direction that is parallel to the front face of the flexible backing will cause the first portion of the first thread to move in a direction that is parallel to the front face of the flexible backing, thereby detaching at least a portion of the flexible backing from the subject's body.

In some embodiments of the third apparatus, the first portion of the first thread is positioned so that an area bounded by the first portion of the first thread and the perimeter of the flexible backing is less than 10% of the total area.

In some embodiments of the third apparatus, the first portion of the first thread is positioned so that an area bounded by the first portion of the first thread and the perimeter of the flexible backing is less than 10% of the total area, the flexible backing has a first corner, and the area bounded by the first portion of the first thread and the perimeter of the flexible backing is located at the first corner.

Optionally, the embodiments described in the previous paragraph further comprise a cord that is affixed to at least one of the substrate and the flexible backing at a position that is less than 3 cm away from the first corner. In these embodiments, the flexibility of the substrate, the flexibility of the flexible backing, the adhesive strength of the first layer of adhesive, the affixation strength of the cord, and the pull strength of the cord are all within respective ranges such that when the first layer of adhesive has been adhered to the subject's body, and the first portion of the first thread has subsequently been moved in a direction that is parallel to the front face of the flexible backing, thereby detaching a portion of the flexible backing from the subject's body, the flexible backing can be peeled away from the subject's body by pulling on the cord.

In some embodiments of the third apparatus, each second portion of the first thread is at least 10 cm long.

Some embodiments of the third apparatus further comprise a second thread having (a) a first portion positioned in front of the flexible backing such that when the first layer of adhesive is adhered to the subject's body, the first portion of the second thread will be disposed between the flexible backing and the subject's body and (b) at least one second portion that extends beyond the perimeter of the flexible backing. In these embodiments, the first and second portions of the second thread are positioned with respect to the flexible backing so that pulling the at least one second portion of the second thread in a direction that is parallel to the front face of the flexible backing will cause the first portion of the second thread to move in a direction that is parallel to the front face of the flexible backing, thereby detaching at least a portion of the flexible backing from the subject's body.

Optionally, in the embodiments described in the previous paragraph, the first portion of the first thread is positioned so that an area bounded by the first portion of the first thread and the perimeter of the flexible backing is less than 10% of the total area, and the first portion of the second thread is positioned so that an area bounded by the first portion of the second thread and the perimeter of the flexible backing is less than 10% of the total area.

Optionally, in the embodiments described in the previous paragraph, the flexible backing has a first corner and a second corner, the area bounded by the first portion of the first thread and the perimeter of the flexible backing is located at the first corner, and the area bounded by the first portion of the second thread and the perimeter of the flexible backing is located at the second corner.

Some embodiments of the third apparatus further comprise a sheet of a polymer material with a dielectric constant of at least 10 disposed in front of the at least one conductive pad, in electrical contact with the at least one conductive pad.

Some embodiments of the third apparatus further comprise a sheet of graphite disposed in front of the at least one conductive pad, in electrical contact with the at least one conductive pad, and the sheet of graphite has a front face and a rear face. Optionally, these embodiments can further comprise a layer of conductive adhesive disposed between the at least one conductive pad and the rear face of the sheet of graphite, and a layer of conductive adhesive disposed on the front face of the sheet of graphite.

Some embodiments of the third apparatus further comprise a cable that is mounted to the substrate, and the cable includes at least one electrical conductor that is electrically connected to the at least one conductive pad.

In some embodiments of the third apparatus, the first thread comprises at least one of nylon and expanded polytetrafluoroethylene.

Another aspect of this Section II is directed to a fourth apparatus for applying an electrical signal to a subject's body. The fourth apparatus comprises a flexible sheet of graphite, a first layer of conductive adhesive, and a first thread. The flexible sheet of graphite has a front face, a rear face, a total area, and an outer perimeter. The first layer of conductive adhesive is disposed on the front face of the sheet of graphite, and the first layer of conductive adhesive is biocompatible and is configured to adhere to the subject's body. The first thread has (a) a first portion positioned in front of the sheet of graphite such that when the first layer of conductive adhesive is adhered to the subject's body, the first portion of the first thread will be disposed between the sheet of graphite and the subject's body and (b) at least one second portion that extends beyond the perimeter of the sheet of graphite. The first and second portions of the first thread are positioned with respect to the sheet of graphite so that pulling the at least one second portion of the first thread in a direction that is parallel to the front face of the sheet of graphite will cause the first portion of the first thread to move in a direction that is parallel to the front face of the sheet of graphite, thereby detaching at least a portion of the sheet of graphite from the subject's body.

In some embodiments of the fourth apparatus, the first portion of the first thread is positioned so that an area bounded by the first portion of the first thread and the perimeter of the sheet of graphite is less than 10% of the total area.

In some embodiments of the fourth apparatus, the first portion of the first thread is positioned so that an area bounded by the first portion of the first thread and the perimeter of the sheet of graphite is less than 10% of the total area, and the sheet of graphite has a first corner, and the area bounded by the first portion of the first thread and the perimeter of the sheet of graphite is located at the first corner.

Optionally, the embodiments described in the previous paragraph further comprise a cord that is affixed to the sheet of graphite at a position that is less than 3 cm away from the first corner. In these embodiments, the flexibility of the sheet of graphite, the adhesive strength of the first layer of conductive adhesive, the affixation strength of the cord, and the pull strength of the cord are all within respective ranges such that when the first layer of conductive adhesive has been adhered to the subject's body, and the first portion of the first thread has subsequently been moved in a direction that is parallel to the front face of the sheet of graphite, thereby detaching a portion of the sheet of graphite from the subject's body, the sheet of graphite can be peeled away from the subject's body by pulling on the cord.

In some embodiments of the fourth apparatus, each second portion of the first thread is at least 10 cm long.

Some embodiments of the fourth apparatus further comprise a second thread having (a) a first portion positioned in front of the sheet of graphite such that when the first layer of conductive adhesive is adhered to the subject's body, the first portion of the second thread will be disposed between the sheet of graphite and the subject's body and (b) at least one second portion that extends beyond the perimeter of the sheet of graphite. In these embodiments, the first and second portions of the second thread are positioned with respect to the sheet of graphite so that pulling the at least one second portion of the second thread in a direction that is parallel to the front face of the sheet of graphite will cause the first portion of the second thread to move in a direction that is parallel to the front face of the sheet of graphite, thereby detaching at least a portion of the sheet of graphite from the subject's body.

Optionally, in the embodiments described in the previous paragraph, the first portion of the first thread is positioned so that an area bounded by the first portion of the first thread and the perimeter of the sheet of graphite is less than 10% of the total area, and the first portion of the second thread is positioned so that an area bounded by the first portion of the second thread and the perimeter of the sheet of graphite is less than 10% of the total area.

Optionally, in the embodiments described in the previous paragraph, the sheet of graphite has a first corner and a second corner, the area bounded by the first portion of the first thread and the perimeter of the sheet of graphite is located at the first corner, and the area bounded by the first portion of the second thread and the perimeter of the sheet of graphite is located at the second corner.

In some embodiments of the fourth apparatus, the first thread comprises at least one of nylon and expanded polytetrafluoroethylene.

### Section III—Conclusion

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an electrical signal to a subject's body, the apparatus comprising:
a flexible substrate having a front face and a rear face;
at least one conductive pad disposed on the front face of the substrate;
a flexible backing affixed to the rear face of the flexible substrate, the flexible backing having an outer perimeter;
a first layer of conductive adhesive or conductive gel disposed in front of the at least one conductive pad, wherein the first layer of conductive adhesive or conductive gel is biocompatible and is configured to adhere to the subject's body; and
a first cable or cord that is affixed to at least one of the substrate and the flexible backing at a position that is less than 3 cm away from the outer perimeter of the flexible backing,
wherein a flexibility of the substrate, a flexibility of the flexible backing, an adhesive strength of the first layer of conductive adhesive or conductive gel, an affixation strength of the first cable or cord, and a pull strength of the first cable or cord are all within respective ranges such that when the first layer of conductive adhesive or conductive gel has been adhered to the subject's body, the flexible backing, the at least one conductive pad, and the flexible substrate can all be peeled away from the subject's body by pulling on the first cable or cord.

2. The apparatus of claim 1, wherein the first cable or cord is affixed to at least one of the substrate and the flexible backing at a position that is less than 1.5 cm away from the outer perimeter of the flexible backing.

3. The apparatus of claim 1, wherein the flexible backing has a first corner, and wherein the first cable or cord is affixed to at least one of the substrate and the flexible backing at a position that is less than 1.5 cm away from the first corner of the flexible backing.

4. The apparatus of claim 1, wherein the flexible backing has a first corner and a second corner,
wherein the first cable or cord is affixed to at least one of the substrate and the flexible backing at a position that is less than 3 cm away from the first corner of the flexible backing, and
wherein the apparatus further comprises a second cord that is affixed to at least one of the substrate and the flexible backing at a position that is less than 3 cm away from the second corner of the flexible backing, and
wherein a flexibility of the substrate, a flexibility of the flexible backing, an adhesive strength of the first layer of conductive adhesive or conductive gel, an affixation strength of the second cord, and a pull strength of the second cord are all within respective ranges such that when the first layer of conductive adhesive or conductive gel has been adhered to the subject's body, the flexible backing, the at least one conductive pad, and the flexible substrate can all be peeled away from the subject's body by pulling on the second cord.

5. The apparatus of claim 1, wherein the first cable or cord is affixed to the flexible backing.

6. The apparatus of claim 1, wherein the first cable or cord is affixed to the substrate.

7. The apparatus of claim 1, further comprising a flexible sheet of conductive material positioned between the at least one conductive pad and the first layer of conductive adhesive or conductive gel, wherein the flexible sheet of conductive material is disposed in electrical contact with both the at least one conductive pad and the first layer of conductive adhesive or conductive gel.

8. The apparatus of claim 1, further comprising a sheet of graphite positioned between the at least one conductive pad and the first layer of conductive adhesive or conductive gel, wherein the sheet of graphite is disposed in electrical contact with both the at least one conductive pad and the first layer of conductive adhesive or conductive gel.

9. The apparatus of claim 1, wherein the first cable or cord is a first cord that is not electrically connected to the at least one conductive pad,
wherein the apparatus further comprises a second cable that is mounted to the substrate in a manner that electrically connects at least one electrical conductor of the second cable to the at least one conductive pad.

10. The apparatus of claim 9, wherein the second cable is attached to the first cord less than 5 cm away from a point at which the first cord is affixed to at least one of the substrate and the flexible backing, and
wherein the second cable and the first cord are configured so that pull forces applied to the second cable in a direction that is parallel to a vector that runs from (a) the point at which the first cord is affixed to at least one of the substrate and the flexible backing to (b) a centroid of the flexible backing are transmitted to the first cord via the second-cable-to-first-cord attachment.

11. The apparatus of claim 1, wherein the first cable or cord is a first cable that includes at least one electrical conductor that is electrically connected to the at least one conductive pad.

12. An apparatus for applying an electrical signal to a subject's body, the apparatus comprising:
a flexible sheet of graphite having a front face, a rear face, and an outer perimeter;
a first layer of conductive adhesive disposed on the front face of the sheet of graphite, wherein the first layer of conductive adhesive is biocompatible and is configured to adhere to the subject's body; and
a first cord that is affixed to the sheet of graphite at a position that is less than 3 cm away from the outer perimeter of the sheet of graphite,
wherein a flexibility of the sheet of graphite, an adhesive strength of the first layer of conductive adhesive, an affixation strength of the first cord, and a pull strength of the first cord are all within respective ranges such that when the first layer of conductive adhesive has been adhered to the subject's body, the sheet of graphite can be peeled away from the subject's body by pulling on the first cord.

13. The apparatus of claim 12, wherein the first cord is affixed to the sheet of graphite at a position that is less than 1.5 cm away from the outer perimeter of the sheet of graphite.

14. The apparatus of claim 12, wherein the sheet of graphite has a first corner, and wherein the first cord is affixed to the sheet of graphite at a position that is less than 1.5 cm away from the first corner of the sheet of graphite.

15. The apparatus of claim 12, wherein the sheet of graphite has a first corner and a second corner,
wherein the first cord is affixed to the sheet of graphite at a position that is less than 3 cm away from the first corner of the sheet of graphite, and
wherein the apparatus further comprises a second cord that is affixed to the sheet of graphite at a position that is less than 3 cm away from the second corner of the sheet of graphite, and
wherein a flexibility of the sheet of graphite, an adhesive strength of the first layer of conductive adhesive, an affixation strength of the second cord, and a pull strength of the second cord are all within respective ranges such that when the first layer of conductive adhesive has been adhered to the subject's body, the sheet of graphite can be peeled away from the subject's body by pulling on the second cord.

16. The apparatus of claim 15, wherein the first cord is affixed to the sheet of graphite at a position that is less than 1.5 cm away from the first corner of the sheet of graphite, and wherein the second cord is affixed to the sheet of graphite at a position that is less than 1.5 cm away from the second corner of the sheet of graphite.

17. The apparatus of claim 12, further comprising a grid of reinforcing threads that is bonded to the sheet of graphite, wherein the first cord is affixed to the grid of reinforcing threads, and wherein the first cord is affixed to the sheet of graphite indirectly via the grid of reinforcing threads.

18. A method of removing an assembly that has previously been affixed to a surface of a subject's body, wherein the assembly includes a flexible patch having a front surface that is coated with a conductive adhesive and a first cable or cord affixed to a first corner of the flexible patch, the method comprising:
pulling the first cable or cord in a direction that deviates by <30° from parallel to the surface of the subject's body so that a first portion of the flexible patch bends back on itself, beginning at the first corner and continuing until the flexible patch has peeled away from the surface of the subject's body, wherein the first corner and a centroid of the flexible patch get closer to each other during an initial phase of motion.

19. The method of claim 18, wherein the assembly includes a second cord affixed to a second corner of the flexible patch, and
wherein the method further comprises pulling the second cord in a direction that deviates by <30° from parallel to the surface of the subject's body so that a second portion of the flexible patch bends back on itself, beginning at the second corner and continuing until the flexible patch has peeled away from the surface of the subject's body, wherein the second corner and the centroid get closer to each other during the initial phase of motion.

20. The method of claim 18, wherein the first cable or cord is pulled in a direction that deviates by <10° from parallel to the surface of the subject's body.
